(19) United States
(12) Patent Application Publication
(10) Pub. No.: US 2013/0013037 A1
(43) Pub. Date: Jan. 10, 2013

(54) Method of Inducing Thermogenesis by Manipulating the Thermoregulatory Status of a Human

(76) Inventor: Jason K. Adams, Ethel, LA (US)

(21) Appl. No.: 13/543,632

(22) Filed: Jul. 6, 2012

Related U.S. Application Data
(60) Provisional application No. 61/506,488, filed on Jul. 11, 2011.
(60) Provisional application No. 61/505,931, filed on Jul. 8, 2011.

(51) Int. Cl. A61F 7/00

(52) U.S. Cl. 607/114

(57) ABSTRACT

This invention provides methods and devices for decreasing the core temperature of a human to cause weight loss. The methods involve contacting the body or a portion of the body with a cooling medium in a sealable chamber to induce a decrease in the core temperature.

### REFERENCE TO RELATED APPLICATIONS

Claim is hereby made to the benefit of the priority of co-pending U.S. Provisional Patent Application No. 61/505,931, filed on Jul. 8, 2011, and U.S. Provisional Patent Application No. 61/506,488, filed on Jul. 11, 2011, the disclosures of which are incorporated herein by reference.

### TECHNICAL FIELD

This invention relates to manipulating the thermoregulatory response of a human to induce thermogenesis.

### BACKGROUND

Conventional weight loss in humans is achieved by creating a caloric net loss in the human body. This caloric net loss can be accomplished through restricted caloric intake or by increased caloric output through activity such as exercise. When humans undertake exercise, they convert stored calories into thermal energy which disperses from the body. It has long been recognized that any time a human can produce thermal energy, the daily caloric load of the human body increases. The difficulty lies in the effort required to generate thermal energy via exercise or increased activity.

### SUMMARY OF THE INVENTION

The present invention provides methods for manipulating the thermoregulatory response of a human to induce thermogenesis. The result of this manipulation is caloric usage (caloric net loss) and weight loss. Thus, the methods of this invention create a caloric net loss in the human to which the method is applied without requiring increased activity such as exercise. Advantageously, the methods of the present invention are non-invasive, and can be repeated any number of times. A further advantage of this invention is that the methods generally do not cause a “cold” sensation to the human undergoing the methods described herein.

An embodiment of this invention is a method of achieving weight loss in a human, which method comprises carrying out two or more treatments, wherein each treatment comprises
a) placing a body or a portion of a body of a human in a sealable chamber and into contact with a cooling medium within the chamber;
b) substantially sealing the chamber at about atmospheric pressure;
c) reducing the pressure within the chamber to a lowered pressure; and
d) maintaining the lowered pressure within the chamber while allowing the body or portion of a body to contact the cooling medium for a period of time sufficient to cause a decrease in the core temperature of the human.

These and other embodiments and features of this invention will be still further apparent from the ensuing description, drawing, and appended claims.

### BRIEF DESCRIPTION OF THE DRAWING

FIG. 1 is representation of a sealable chamber for a cooling device that can be used in the practice of this invention.

### FURTHER DETAILED DESCRIPTION OF THE INVENTION

This invention provides a method to increase daily caloric output by reducing the core body temperature of the human employing the method of this invention. This reduction in core body temperature in turn causes the human body to cause thermogenesis to make up for the net lost of heat. Humans are homeothermic, which means that human bodies regulate their core temperature to a particular value, generally near or at about 98.6° F. (37° C.). Calories in a human body are burned by thermogenesis until the body attains or re-attains its core temperature (e.g., 98.6° F.), and the calories burned can include calories stored as fat. Every calorie of energy removed by cooling the body must be replaced, and can be replaced from energy (fat) stores in the human body.

As mentioned above, the present invention provides methods of achieving weight loss in humans. The methods comprise carrying out two or more treatments. Treatments may be repeated, for example, a) once or more daily; b) twice or more weekly; d) twice or more monthly, or e) twice or more yearly. Preferably, treatments are repeated on relatively frequent bases, such as once or more daily or twice or more weekly, in order to achieve weight loss over shorter periods of time.

Each treatment can be carried out for a period of time as short as about one minute or as long as about 1440 minutes (24 hours); preferred times for each treatment are about 5 minutes or more, and preferably range from about 5 minutes to about 500 minutes. While it is possible to perform the methods of the invention for 24 hours a day, it may not be practical to do so.

Between each treatment, enough time preferably elapses to allow the body of the human to achieve normal thermal regulation. This time will vary based on various factors, including, but not limited to, i) age of the human, ii) weight of the human, and/or iii) current health condition of the human.

In each treatment, a body or a portion of a body of a human is placed in a sealable chamber and into contact with a cooling medium within the chamber. As used throughout this document, the term “portion of a body” means any part of a body, including all or part of one or more limbs, which can include the head. Preferably, a portion of a body is one limb or a portion of one limb. Different portions of a body can be used in each treatment, as desired, since the effect of the methods described herein are systemic.

Sufficient contact between the body or portion of the body and the cooling medium allows for sufficient thermal exchange to induce a net negative thermal caloric load (a decrease in core body temperature) which in turn induces thermogenesis. The extent of contact that is sufficient varies, depending upon the desired rate of thermogenesis. Thermal energy transfer efficiency generally increases as the surface area in contact with the cooling medium increases.

Once the body or portion of a body is placed in the sealable chamber, the chamber is substantially sealed at about ambient pressure by increasing the pressure on the seal bladder of the chamber. Increasing the pressure on the seal bladder provides a suitable airtight seal on the body or portion of a body.

Once an airtight seal is achieved, the pressure within the chamber is reduced to a lowered pressure, and a lowered pressure is maintained in the chamber during the treatment. As used throughout this document, the term “lowered pressure” means a pressure below the ambient (typically atmospheric) pressure. The lowered pressure is sometimes referred to as “negative net pressure.” The pressure within the chamber is usually reduced by a vacuum-inducing mechanism or device (e.g., a vacuum pump) and exposes the body or portion thereof to a lowered pressure, which lowered pressure is preferably in the range of about 20 mmHg (2.66×10³ Pa or 0.385 psi) to about 85 mmHg (1.13×10⁴ Pa or 1.64 psi) less than ambient pressure. One or more different lowered pressures may be employed in the same treatment step.

Once the desired lowered pressure is reached, the body or portion of the body exposed to the lowered pressure undergoes vasodilation. Vasodilation ensures that blood moves to the surface of the body or portion of the body in the chamber and allows effective thermal exchange to occur. The body or portion of a body is allowed to contact the cooling medium for a period of time sufficient to cause a decrease in the core body temperature of the human.

The cooling medium in contact with the body or portion of a body is at one or more temperatures in the range of about 40° F. (4° C.) to about 95° F. (35° C.), preferably in the range of about 55° F. (13° C.) to about 85° F. (29° C.), more preferably in the range of about 65° F. (18° C.) to about 75° F. (24° C.), and even more preferably about 70° F. (21° C.). Once the chamber is sealed, the lowered pressure is attained, and the cooling medium is in contact with the body or portion thereof, thermal transfer of energy takes place, and thermogenesis begins (or the rate of thermogenesis increases) and continues as long as needed for the human undergoing the method to achieve the desired core body temperature, if the described conditions provided by the methods of this invention are maintained. The thermogenesis induced by the methods of this invention can stimulate fat (weight) loss.

Preferably, the period of time sufficient to cause a decrease in the core temperature of the human triggers a detectable increase in thermogenesis or a detectable loss in temperature of a human. A detectable increase in thermogenesis may be measured in a variety of ways. Some examples of suitable methods include:
tracking the total weight of the human over a period of time, such as a day, a week, a month, or a year, while calculating caloric intake;
measuring the human's ability to maintain a static body temperature under an increased negative thermal load; and
measuring the temperature of a circulating liquid before the circulating liquid enters the cooling medium, and comparing this value to the temperature of the circulating liquid after it exits the cooling medium. The temperature difference of the circulating liquid is calculated and compared to the difference in the core body temperature of the human undergoing the method before, during, and after treatment to calculate thermogenesis and increased heat production of the human.

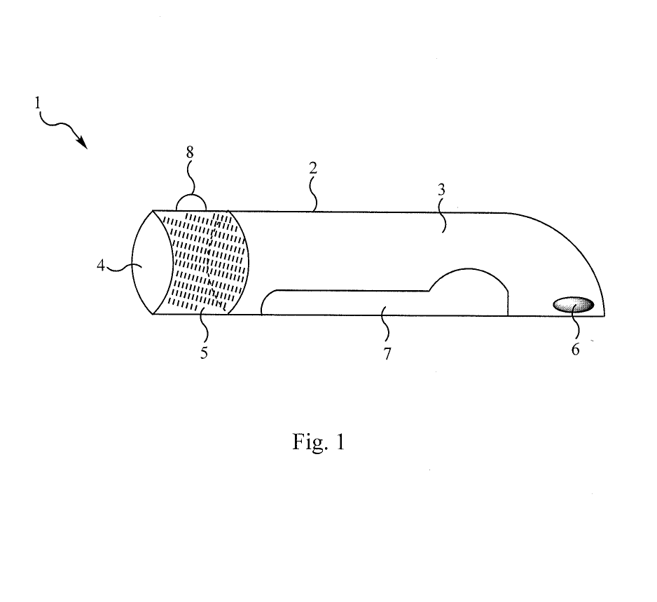

Another embodiment of this invention is a cooling device for humans to decrease their core body temperature as a method to increase caloric net loss. An example of a device that can be used to carry out a preferred method in accordance with this invention is illustrated in FIG. 1. The cooling device 1 comprises a sealable chamber. The sealable chamber 2 is sized and configured to enclose a body or a portion of a body of a human (not shown). Further, the sealable chamber defines an interior 3 and also defines an opening 4, about which opening 4 is placed a seal bladder 5. The seal bladder 5 is configured to seal the sealable chamber 1, and the seal bladder 5 can be made from e.g., silicone. Cooling device 1 also has a valve 6 in fluid connection with the interior 3 of the sealable chamber 2, and valve 6 is adapted for connection to a vacuum-inducing device. Through valve 6, a vacuum is applied to reduce the pressure in sealable chamber 2 to a lowered pressure value. Lowered pressure values and the preferences therefor are as described above. A cooling medium 7 is present in the interior 3 of the sealable chamber 1. As shown in FIG. 1, cooling medium 7 has a Peltier junction cooling surface. The seal bladder 5 can comprise a pressure release valve 8.

Still referring to FIG. 1, increasing the pressure on seal bladder 3 provides a suitable airtight seal around a body or portion of a body of a human (not shown) placed in the sealable chamber 2. Once an airtight seal is achieved, a vacuum is applied via valve 6, creating a lowered pressure in the interior 3 of sealable chamber 2, which exposes the body or portion of a body of a human therein to a lowered pressure. While the body or portion of the body is exposed to the lowered pressure, the body or portion of the body is also exposed to the cooling medium 7, for which the temperatures and preferences are as described above. Once these conditions are achieved, thermal transfer of energy takes place, inducing thermogenesis, which continues as long as the device is in use.

The sealable chamber may be comprised of a sealable, enclosable volume large enough to encompass a body or a portion of a body of a human. Suitable non-limiting examples of the sealable chamber can include:

mobile and powered, providing its own negative pressure and cooling capabilities;
mobile and powered, providing its own negative pressure capabilities but relying on a external cooling device such as an ice pack or other device that requires external cooling abilities such as a refrigerator or freezer;
stationary, relying on a battery and/or an external power source for cooling and pressure reduction;
mobile and un-powered, using human power via a manual vacuum-inducing mechanism or device for its pressure reduction and relying on a external cooling device such as an icepack or other device that requires external cooling abilities such as a refrigerator or freezer;
automatic, using methods or controls which automatically manipulate the cooling medium temperature and lowered pressure in the chamber to achieve the optimum thermal exchange; and
manual, relying on the user to manipulate the controls or equipment to maintain optimum conditions to achieve the optimum thermal exchange.

The sealing surface of the seal bladder between the body or portion of a body and the enclosure will provide sufficient integrity to maintain a reduced pressure environment around the body part for a sufficient length of time to achieve vasodilation of the blood vessels in the body part and allow sufficient thermal energy exchange to induce thermogenesis or increase the rate of thermogenesis. In preferred embodiments, the seal bladder comprises a pressure release valve.

The cooling medium may be, for example, metal, gel, laser, magnetic, chemical, or another thermally conductive material that can exchange heat with:
a thermo-electric cooling element, which is a device that creates a reduced temperature on its surface when a voltage is applied to the cooling element. The cooling effect measured on the surface of the cooling element can be increased or decreased by varying the applied voltage);
a circulating liquid medium, which can be used to transfer thermal energy from the body or portion of the body in contact with the surface of the cooling medium. This medium would then pass through a heat exchanging device removing the thermal energy from the circulating liquid medium;
a heat pump refrigeration device;
a vapor compression refrigeration device; or
a vapor absorption refrigeration device.

Pressure reduction is achieved via a valve that permits application of a vacuum until the desired lowered pressure is attained. As described above, the lowered pressure in the sealable chamber can be achieved through a variety of means of applying a vacuum.

Other benefits of the practice of this invention may include relieving stress, decreasing and/or curing depression, increased sense of wellbeing, boosting immune response, reducing the effects of oxidative stress, reducing fatigue in those who have “chronic fatigue syndrome”, improving the prognosis of patients with chronic heart failure, and stimulating anti-tumor immunity for some types of cancers.

The invention may comprise, consist, or consist essentially of the materials and/or procedures recited herein.

Except as may be expressly otherwise indicated, the article “a” or “an” if and as used herein is not intended to limit, and should not be construed as limiting, the description or a claim to a single element to which the article refers. Rather, the article “a” or “an” if and as used herein is intended to cover one or more such elements, unless the text expressly indicates otherwise.

Each and every patent or other publication or published document referred to in any portion of this specification is incorporated in tow into this disclosure by reference, as if fully set forth herein.

This invention is susceptible to considerable variation in its practice. Therefore the foregoing description is not intended to limit, and should not be construed as limiting, the invention to the particular exemplifications presented hereinabove.

That which is claimed is:

1. A method of achieving weight loss in a human, which method comprises carrying out two or more treatments, wherein each treatment comprises
a) placing a body or a portion of a body of a human in a sealable chamber and into contact with a cooling medium within the chamber;
b) substantially sealing the chamber at about atmospheric pressure;
c) reducing the pressure within the chamber to a lowered pressure; and
d) maintaining the lowered pressure within the chamber while allowing the body or portion of a body to contact the cooling medium for a period of time sufficient to cause a decrease in the core temperature of the human.

2. A method as in claim 1 wherein said treatments are repeated once or more daily or twice or more weekly.

3. A method as in claim 1 wherein said cooling medium is at a temperature in the range of about 40° F. to about 95° F.

4. A method as in claim 1 wherein said cooling medium is at a temperature in the range of about 55° F. to about 85° F.

5. A method as in claim 1 wherein said lowered pressure is in the range of about 20 mmHg to about 85 mmHg.

6. A method as in claim 1 wherein the period of time is about one minute or more.

7. A method as in claim 1 wherein the period of time is in the range of about 5 minutes to about 500 minutes.

8. A method as in claim 1 wherein the method is conducted with one or more of the following conditions:
said cooling medium is at a temperature in the range of about 65° F. to about 75° F.;
said lowered pressure is in the range of about 20 mmHg to about 85 mmHg;
the period of time is about 5 minutes or more.

9. A method as in claim 8 wherein the method is conducted with all of the conditions listed.

10. A method as in claim 1 wherein a cooling device is employed, the device comprising:
a sealable chamber sized and configured to enclose a body or a portion of a body of a human, defining an interior and defining an opening about which opening is placed a seal bladder, said seal bladder being configured to seal the sealable chamber;
a valve in fluid connection with the interior of the sealable chamber, said valve adapted for connection to a vacuum-inducing device; and
a cooling medium present in the interior of the sealable chamber.

11. A method as in claim 10 wherein the seal bladder comprises a pressure release valve.

12. A cooling device for decreasing the core temperature of a human, which device comprises
a sealable chamber sized and configured to enclose a body or a portion of a body of a human, defining an interior and defining an opening about which opening is placed a seal bladder, said seal bladder being configured to seal the sealable chamber;
a valve in fluid connection with the interior of the sealable chamber, said valve adapted for connection to a vacuum-inducing device; and
a cooling medium present in the interior of the sealable chamber.

13. A device as in claim 12 wherein the seal bladder comprises a pressure release valve.
